(19) United States
(12) Patent Application Publication
(10) Pub. No.: US 2025/0090787 A1
(43) Pub. Date: Mar. 20, 2025

(54) BREATHING TUBE WITH A MORE EASILY INTERCHANGEABLE BREATHING-GAS FILTER

(71) Applicant: HAMILTON MEDICAL AG, Bonaduz (CH)

(72) Inventors: Marco Gruber, Davos Wolfgang (CH); Regula Hidber, Wangs (CH); Jan Hunger, Chur (CH); Ingo Schwaiger, Chur (CH)

(73) Assignee: HAMILTON MEDICAL AG, Bonaduz (CH)

(21) Appl. No.: 17/626,769

(22) PCT Filed: Mar. 31, 2020
(86) PCT No.: PCT/EP2020/059088

§ 371 Date: Jan. 12, 2022

(30) Foreign Application Priority Data
Jul. 19, 2019 (DE) 10 2019 119 674.4

(51) Int. Cl. A61M 16/10; A61M 16/00; A61M 16/04

(52) U.S. Cl. CPC A61M 16/1055; A61M 16/0003; A61M 16/022; A61M 16/04; A61M 16/1075; A61M 2016/0033

(57) ABSTRACT

A breathing tube which is designed for channelling inspiratory and/or expiratory breathing gas during at least partial artificial respiration of a patient, defines a virtual breathing-gas-flow path, which is imagined to pass centrally through the tube and along which the breathing tube channels the breathing gas. The breathing tube has a filter carrier in which is formed a filter-accommodating space, which has the breathing-gas-flow path passing through it and in which a breathing-gas filter, which can have breathing gas flowing through it, can be accommodated in an interchangeable manner. The filter carrier has a wall which encloses the filter-accommodating space at a radial distance from the virtual breathing-gas-flow path and has an introduction opening, which is arranged at a radial distance from the virtual breathing-gas-flow path and through which the breathing-gas filter can be introduced into the filter-accommodating space and can be moved into its operationally ready operating position.

The present invention relates to a respiratory line which is designed for conveying inspiratory or/and expiratory breathing gas during at least partial artificial respiration of a patient, wherein the respiratory line defines a conceived as extending imaginary virtual breathing gas flow path, which is conceived as extending centrally through the respiratory line and along which the respiratory line conveys the breathing gas, wherein the respiratory line has a filter carrier in which a filter receiving space through which a breathing gas flow path passes is formed, in which a breathing gas filter, through which breathing gas can flow, can be replaceably accommodated.

Such a respiratory line is known from WO 2013/127474 A1. Respiratory lines are used for conveying inspiratory or/and expiratory breathing gas on respiratory devices for at least supportive artificial respiration of human or animal patients, in order to convey inspiratory breathing gas from a breathing gas source to the patient or/and in order to convey expiratory breathing gas away from the patient.

In the routing of the respiratory line, breathing gas filters are sometimes arranged, for example, in order to remove particles or/and droplets which are harmful to the patient or undesired from the inspiratory breathing gas or/and in order to remove pathogens exhaled by the patient from the expiratory breathing gas.

Since a breathing gas filter which has been used once naturally has only a limited lifespan and must then be replaced by a fresh or at least a less used breathing gas filter, a filter carrier is provided on the known respiratory line, which enables a replacement of a used breathing gas filter by an unused breathing gas filter.

A disadvantage of the known respiratory line is that the breathing gas flow has to be interrupted for changing the breathing gas filter, since, for a replacement of the breathing gas filter, the respiratory line has to be physically interrupted or opened. For the duration of a filter change, artificial respiration can thus not occur.

Also known from U.S. Pat. No. 3,556,097 and GB 1 294 307 A are respiratory lines with a replaceable breathing gas filter, wherein in each case the breathing gas flow must be interrupted for the filter replacement.

Therefore, the underlying aim of the present invention is to improve the respiratory line mentioned at the start in such a manner that it allows changing the breathing gas filter without interruption of a breathing gas flow in the respiratory line.

This aim is achieved according to the invention by a respiratory line of the type mentioned at the start, in which the filter carrier has a wall surrounding the filter receiving space with radial spacing from the virtual breathing gas flow path, having an introduction opening arranged with radial spacing from the virtual respiration flow path, through which the breathing gas filter can be introduced into the filter receiving space and be brought into its operational position.

While, in the respiratory line of WO 2013/127474 A1, an introduction opening is only formed when two respiratory line sections are disconnected in the region of the filter receiving space, wherein the introduction opening is passed through by the virtual breathing gas flow path, that is to say without radial spacing from the virtual breathing gas flow path, in the respiratory line according to the invention, the introduction opening located at a distance radially from the virtual breathing gas flow path can exist permanently, independently of the operating state of the remaining components of the respiratory line or even of the respiratory device, so that a breathing gas filter can be introduced into the filter receiving space at any time.

With the introduction movement, the breathing gas filter can be moved from the introduction opening closer to the virtual breathing gas flow path and thus be arranged, operational, in the breathing gas flow of the respiratory line in such a manner that said breathing gas flow can flow through it. In the operational position, the virtual breathing gas flow path passes through the breathing gas filter, so that, in the region of the filter receiving space, the breathing gas flow hits the breathing gas filter and flows through it making use of its filtration effect.

A change of the filter receiving space or of the wall surrounding the filter receiving space is not necessary for the introduction of the breathing gas filter into the filter receiving space. Thus, during introduction of the breathing gas filter into its operating position in the filter receiving space, the respiration operation, in which the respiratory line participates at the given time, can be maintained.

Between the time of removal of a used-up breathing gas filter and the time of an arrangement of a new breathing gas filter in its operating position, the breathing gas is in fact unfiltered in the respiratory line. However, this is a considerably reduced disadvantage in comparison to a complete interruption of artificial respiration.

In principle, it is conceivable that the breathing gas filter need only be introduced skillfully by the given working operator through the introduction opening into the filter receiving space and into the operating position. However, for the fastest, most reliable and targeted filter replacement possible, with the shortest possible interruption of only the filter action but not of the artificial respiration itself, it is particularly advantageous if the filter carrier has a guiding structure which guides an introduction movement of the breathing gas filter from the introduction opening into the filter receiving space. Thus, it can be sufficient for an operator merely to push a new breathing gas filter, arranged correctly on the introduction opening, approximately in the correct direction. The guiding structure on the filter carrier then ensures that the new breathing gas filter correctly performs a desired movement into the filter receiving space. Preferably, the guiding structure ensures not only a desired movement of the breathing gas filter into the filter receiving space but also, in a preferred development of the present invention, a guiding of the movement of the breathing gas filter into its operating position.

The guiding structure can be formed by a channel passing through the filter carrier, which is preferably formed by wall sections of the filter carrier, which surround the filter receiving space. It is also conceivable, in an embodiment which is less preferable due to its complexity, that the filter carrier has at least one guide rail or/and guide groove forming the guiding structure.

Preferably, the reaching of the operating position of the breathing gas filter can be perceived by an operator pushing, in particular manually, the breathing gas filter into the filter receiving space, for example, via a haptic or/and acoustic or/and optical signal. The haptic or/and acoustic signal can be achieved by a locking of the breathing gas filter in the operating position. Therefore, it is preferable that at least one locking formation is arranged in the filter receiving space, in particular on the guiding structure, which interacts with a filter-side locking counter-formation for locking the breathing gas filter in the operating position.

An optical signal can be achieved by an appropriate color or/and shape design of the breathing gas filter, in that, for example, a color or/and shape limit on the breathing gas filter assumes a predetermined relative position, characterizing the reaching of the operating position, relative to a reference section in the region of the filter receiving space, in particular in the region of the introduction opening, such as, for example, on an edge of the introduction opening.

In the present application, when mention is made of a “new” breathing gas filter, this does not necessarily mean a brand-new breathing gas filter, although, as a rule, this is the case. A “new” breathing gas filter is understood to designate any breathing gas filter which is introduced as first breathing gas filter into the filter receiving space or which is to replace a more used-up breathing gas filter in the filter receiving space.

In principle, it is conceivable that a used-up breathing gas filter, by reversal of the introduction movement through the introduction opening, can be removed again from the filter receiving space. However, this is not advantageous due to the existing contamination risk. This is the case, since a potentially contaminated used-up breathing gas filter is moved through an introduction opening through which subsequently a clean new filter is to be introduced into the filter receiving space. A reduction of this contamination risk can be achieved in that the wall of the filter carrier has a removal opening different from the introduction opening and arranged with radial spacing from the virtual breathing gas flow path, through which a breathing gas filter arranged in the filter receiving space can be removed from the filter receiving space. Thus, the removal of a breathing gas filter from the filter receiving space advantageously can occur through a different opening from that used for the introduction of a breathing gas filter into the filter receiving space.

The removal of a used-up breathing gas filter, which, at the beginning of the removal procedure, is often only accessible with a possibly very small physical section for the initiation of the removal procedure, can be facilitated in that the guiding structure is arranged between the introduction opening and the removal opening, so that it also guides a removal movement of a breathing gas filter from the filter receiving space. This enables a particularly advantageous operation, according to which a used-up breathing gas filter can be dislodged from the filter receiving space by the new breathing gas filter. With the introduction of the new breathing gas filter, the used-up breathing gas filter can be shifted or in general be moved out of the filter receiving space by the new breathing gas filter.

For this purpose, the new breathing gas filter preferably has, at least on its leading end region in the direction of introduction, a reinforcement formation such as, for example, a reinforcing shoulder, so that it is possible to exert sufficient force on the used-up breathing gas filter arranged in the filter receiving region to dislodge it.

Since, for preventing errors during the respiratory operation, breathing gas filters of preferably identical design are used in a temporal succession, the breathing gas filter also has, on its trailing end region in the direction of introduction, a reinforcement formation such as, for example, a reinforcing shoulder, so that it is possible to withstand the dislodging force of the new breathing gas filter during its introduction. This is the case, since the purpose of the force transmission between new unused breathing gas filter is not the deformation of at least one of the two filters, but their desired movement. Therefore, a breathing gas filter preferably has a filter material through which breathing gas can flow, in particular a flat filter material, which is surrounded by a frame which is rigid in comparison to the filter material. Preferably, as circumferential frame, the frame has a central passage opening, by which the breathing gas flows during respiratory operation through the filter material surrounded by the frame.

Although, fundamentally, complex movement paths for introducing or/and removing a breathing gas filter are conceivable and even advantageous in order to avoid an unwanted removal of a breathing gas filter from its operating position, complex movement paths can require high displacement forces in order to bring about the desired movement. A filter replacement can then advantageously be carried out with a small displacement force if the breathing gas filter can be moved along a bend-free guide path defined by the guiding structure, through the introduction opening, toward the virtual breathing gas flow path, into the filter receiving space and into the operating position as well as out of the operating position, away from the virtual breathing gas flow path, out of the filter receiving space through the removal opening. Preferably, this bend-free guide path is a straight guide path. Then, the introduction opening and the removal opening can be formed on opposite sides of the wall of the filter receiving space along the guide path.

In order to reduce the aforementioned contamination risk, the filter carrier can be designed to allow a movement of a breathing gas filter into the filter receiving space and out of said filter receiving space only in exactly one direction. This applies particularly to the guiding structure, by which the movability of the breathing gas filter into the filter carrier and out of said filter carrier can be influenced particularly effectively. For this purpose, the filter carrier and in particular the guiding structure on the filter carrier can have an inhibiting formation which allows a movement of the breathing gas filter through the introduction opening into the filter receiving space and inhibits an oppositely directed movement of the breathing gas filter through the introduction opening. The inhibiting formation can be designed in the manner of a latch lock.

In principle, the inhibiting formation can generate the desired inhibiting action by acting directly on the porous filter material or a correspondingly deformable frame material of an aforementioned frame. For example, a toothing formation inclined in the direction of the allowed movement can be provided as inhibiting formation on the filter carrier or on the guiding structure, so that, during movement in the allowed movement direction, the filter or frame material slides away under the toothing formation, and the toothing formation penetrates into the filter or frame material when an attempt is made to move the breathing gas filter in the inhibited movement direction.

A particularly reliable unidirectional movement inhibition can be achieved in that the breathing gas filter has an inhibiting counter-formation which cooperates with the inhibiting formation of the filter carrier to achieve the unidirectional movability of the breathing gas filter. Preferably, the inhibiting counter-formation cooperates with the inhibiting formation in a positive-locking manner. By the design of an inhibiting counter-formation which has to cooperate with the inhibiting formation provided on the filter carrier or on the guiding structure, the risk of arranging an incorrect breathing gas filter in a filter carrier can moreover be considerably reduced. In the case of a different or lacking inhibiting counter-formation, the introduction of an incorrect new breathing gas filter becomes immediately perceptible to the given working operator.

In a particularly effective and therefore preferable embodiment, the inhibiting formation and the inhibiting counter-formation form a plurality of unidirectionally surmountable catches located one after the other along the allowed movement direction. For example, the inhibiting formation and the inhibiting counter-formation can each have a sawtooth formation formed by a sufficiently elastic material and running along the guide path. The sloping surfaces of the two sawtooth formations, which are less strongly inclined with respect to the guide path or the movement direction, can slide in one direction past one another, whereas in the opposite direction the sloping surfaces of the two sawtooth formations, which are more strongly inclined with respect to the guide path or the movement direction, form a physical movement barrier which cannot be surmounted without destruction.

The aforementioned reinforcement formations, in particular shoulders, particularly preferably the circumferential frame, are or is preferably gas impermeable, so that the breathing gas filter provides a defined passage opening for a throughflow of breathing gas. Preferably, the aforementioned frame or a reinforcing shoulder of the breathing gas filter, in the operating position of the breathing gas filter, is used for sealing the introduction opening. Moreover, preferably, the aforementioned frame or a reinforcing shoulder of the breathing gas filter, in the operating position of the breathing gas filter, is also used for sealing the removal opening. Thus, simply by arranging the breathing gas filter in its operating position in the filter carrier or in the filter receiving space, it can be ensured that the filter receiving space which is passed through by flowing breathing gas is separated from the environmental atmosphere surrounding the filter carrier. According to a preferred development of the present invention, in the edge region of the introduction opening or/and of the removal opening, a sealing lip or in general another sealing formation can be provided, which, in the operating position of the breathing gas filter, sealingly lies against the reinforcing shoulder or the frame of the breathing gas filter. As described in this section, the frame or at least the shoulder can form a flow barrier against an undesired throughflow of breathing gas through the introduction opening or/and the removal opening. However, for this purpose, it is necessary that the shoulders or frames forming the flow barrier are designed to be gas impermeable.

The wall having the introduction opening or/and the removal opening can be part of a housing of the filter carrier surrounding the filter receiving space in the operational state. For purposes of cleaning or/and maintenance, the housing as a dividable housing can have at least two housing portions which can be moved apart or closer together. Thus, it should not be excluded that the housing of the filter carrier can be disassembled in at least two portions. However, according to the invention, such a disassembly should not be necessary for a breathing gas filter change.

In principle, the filter carrier must allow the throughflow of breathing gas. For this purpose, on both sides of the filter receiving space, the filter carrier can have flow-conveying line sections, for example, connection nozzles for the respective connection of a breathing tube. Frequently, it is desirable to be able to influence the temperature of the breathing gas filter in its operating position, in particular to be able to heat the breathing gas filter in its operating position. For the arrangement of a filter heater on the filter carrier, aligned line sections on both sides of the filter receiving space can be disadvantageous, since they can limit a heating surface of a heating device which can be detachably arranged on the filter carrier. For achieving the largest possible heating surface with simultaneous separability of the filter carrier from a heating device supplying the heat output, it is instead advantageous if the filter carrier, on both sides of the filter receiving space, has flow-conveying line sections, of which respective associated sections of the virtual breathing gas flow path enclose an angle with one another.

In order to influence the temperature of the breathing gas filter, the respiratory line can have a filter heater as the aforementioned heating device, which at least partially surrounds the filter carrier in the operational state.

In order to be able to remove the filter carrier from the filter heater for repair or/and maintenance, the filter heater can have a filter carrier accommodation designed as a component separate from the filter carrier, into which the filter carrier can be inserted and from which an inserted filter carrier can be removed. The filter carrier here can be removed from the filter heater without the breathing gas flow having to be interrupted or the respiratory line having to be opened for this purpose.

Here, a filter replacement can be carried out even if the filter carrier is inserted in the filter carrier accommodation of the filter heater, if, according to an advantageous development of the present invention, the filter heater has a wall with a passage opening which, in the operational state, is aligned with the introduction opening in such a manner that a breathing gas filter can be introduced through the passage opening into the introduction opening and thus into the filter carrier. The same also applies likewise, mutatis mutandis, to the removal opening. According to this advantageous development, a wall of the filter heater consequently should have a passage opening which is aligned with the removal opening in the operational state.

In principle, the breathing gas filter can be stored in any desired manner until its use and then be moved close to the filter carrier. Preferably, the breathing gas filter is in packaged form before its introduction into the filter receiving space, in order to protect the breathing gas filter from outside influences, in particular soiling. As already stated above, the breathing gas can be introduced along an introduction movement path into the filter receiving space. The introduction movement path is preferably the aforementioned guide path defined by the guiding structure. For the protection against external influences, the breathing gas filter, in its starting state before its introduction into the filter receiving space, is preferably accommodated in a sleeve, wherein the sleeve moreover is preferably designed to contribute to the introduction of the breathing gas filter into the filter receiving space.

In order to not only protect the breathing gas filter during its storage and transport from outside influences but also to be able to introduce the breathing gas filter into the filter receiving space under the most hygienic conditions possible, the sleeve preferably has a coupling formation. Likewise, depending on whether the new breathing gas filter is introduced directly through the introduction opening of the filter carrier or before that through a passage opening of a filter heater into the filter receiving space, either the filter carrier or the filter heater has a coupling counter-formation which can be coupled to the coupling formation.

According to a preferred development of the present invention, the coupling formation can be coupled to the coupling counter-formation of the filter carrier or of the filter heater in such a manner that, in a transfer state, in which an access opening of the sleeve, which is designed for releasing the breathing gas filter from the sleeve, is aligned with the introduction opening along the introduction movement path, the sleeve is held on the coupling counter-formation. Thus, not only can the sleeve with a new breathing gas filter be temporarily arranged on the filter carrier or on the filter heater without having to be continually held, but also, due to the engagement interaction of coupling formation and coupling counter-formation, at least in the transfer state, the sleeve can cover the introduction opening and possibly also the passage opening. As a result, the filter receiving space is also protected by the sleeve coupled via its coupling formation to the filter carrier or to the filter heater against outside influences such as soiling. When the sleeve is coupled by means of its coupling formation to the coupling counter-formation, the filter carrier can be moved conveniently, without the sleeve being detached thereby from the filter carrier or from the filter heater. This considerably simplifies a filter replacement.

Since in the transfer state the access opening of the sleeve is aligned with the introduction opening and possibly also with the passage opening along the introduction movement path, the new breathing gas filter which is still arranged in the sleeve can be brought particularly simply from the sleeve through the mentioned at least one opening into the filter receiving space. Particularly advantageously, this can occur hygienically without the personnel touching the breathing gas filter and without contact of the breathing gas filter with the outside environment if at least one wall section of the sleeve can be shifted relative to the coupling formation in such a manner that the breathing gas filter can be shifted through the access opening out of the sleeve by displacement the displaceable wall section relative to the coupling formation.

In principle, the displaceable wall section on the remaining sleeve can be designed similarly to a displaceable piston in a cylinder, so that the displaceable wall section can be brought in contact engagement with the breathing gas filter, more precisely with its reinforcing edge bar, more preferably with its frame surrounding the filter material, and can be shifted out of the access opening by moving the wall section. In this case, the breathing gas filter is in the storage state of a breathing gas filter arrangement including the breathing gas filter and the sleeve between the displaceable wall section and the access opening. However, although such an elaborate displaceable wall section is in principle possible, due to the complexity of this design it is not preferable. For reasons relating to the simplest possible production and handling and thus also for reasons relating to lowest possible costs, it is preferable for the sleeve to be designed flexibly at least in sections, in such a manner that the shift which shifts the breathing gas filter through the access opening out of the sleeve is a deformation of the displaceable wall section. At least in its wall section which can be displaced by deformation, the sleeve can be made of a soft elastic plastic such as, for example, a polyolefin or else of a tear-resistant polyethylene terephthalate, formed in particular of a flexible plastic film. However, the sleeve can still be produced out of an elastomer such as, for example, rubber or silicone, so that it can be deformed with small force with larger wall thicknesses. The sleeve can be formed at least in sections as a folded bellows which can be deformed in the direction of the access opening or away from said access opening. In the case of a design of the sleeve as a folded bellows at least in sections, the sleeve can also be made of a stiffer material which, as a flat outer surface having the same thickness as an elastomer sleeve which can be easily deformed by manual handling, can be deformed only with considerably greater force or can no longer be deformed by a manual handling without tool.

Again, for the effective introduction of the breathing gas filter from the interior space of the sleeve, through the introduction opening, into the filter receiving space of the filter carrier, the sleeve is preferably designed so that it can be deformed at least on its end which is at its end far from the coupling formation. The sleeve can be designed so that it can be deformed from this far end to its access opening. Here, “can be deformed” is understood to mean that even a weak individual such as, for example, an untrained elderly female, can perform a deformation without a tool by manual handling.

When it is stated above that the breathing gas filter arrangement, that is to say the sleeve with the breathing gas filter accommodated therein, is coupled by means of the coupling formation of the sleeve in the transfer state to the coupling counter-formation in the above-described manner, this is not intended to mean that the breathing gas filter arrangement is coupled or can be coupled only in the transfer state to the coupling counter-formation, although this should not be excluded. Preferably, the coupling formation and the coupling counter-formation already allow a coupling engagement before reaching the transfer state, whereby the sleeve or the breathing gas filter arrangement, when the coupling engagement has been established, can be shifted relative to the filter carrier along a coupling path into a relative position corresponding to the transfer state. So that the sleeve or the breathing gas filter arrangement in the transfer state can be held with a sufficient holding force by the coupling engagement in the transfer state, the coupling path runs preferably transversely to the introduction movement path of the breathing gas filter when said breathing gas filter is introduced into the filter carrier. As a result, the coupling engagement of coupling formation and coupling counter-formation can form, along the introduction movement path, a protection against lifting off, preferably a positive-locking protection against lifting off because of its greater reliability. Therefore, the coupling engagement of coupling formation and coupling counter-formation is preferably a positive-locking coupling engagement.

The coupling formation can be designed as separate from the remaining sleeve and be connected thereto. This is preferable if a skin of the sleeve surrounding the breathing gas filter is made of a flexible plastic film. Then, the coupling formation can be designed as an inherently stiff component and be connected to the skin, for example, by gluing or fusing.

When the skin of the sleeve is dimensionally stable enough to receive the expected holding forces in the region of the coupling engagement during a filter replacement, the coupling formation can be designed to form a single piece with the skin of the sleeve.

Although it should not be excluded that the sleeve or the breathing gas filter arrangement, starting from an initial coupling engagement, is rotated partially or completely by rotation about an axis of rotation into the transfer state, it is preferable if the sleeve or breathing gas filter arrangement, after establishment of an initial coupling engagement, can be brought by a slight and reliably guidable translational shifting movement into the transfer state. For simple handling, the sleeve or breathing gas filter arrangement can preferably be shifted just by a translational movement into the transfer state.

For guiding the movement of the sleeve or of the breathing gas filter arrangement along its coupling path, with coupling engagement established between coupling formation and coupling counter-formation, one formation, of coupling formation and coupling counter-formation, can include at least one protrusion, and the respective other formation, of coupling formation and coupling counter-formation, can moreover have at least one groove, wherein, at least in the transfer state, the at least one protrusion preferably engages already during the relative movement along the coupling path in the at least one groove. Thus, a groove surrounding a protrusion in circumferential direction around the virtual path can lead to a movement of the sleeve or of the breathing gas filter arrangement along the coupling path. For this purpose, the groove runs along the coupling path. On the other hand, the groove surrounding the protrusion can secure the sleeve or the breathing gas filter arrangement orthogonally to the coupling path against lifting off from the filter carrier.

Preferably, the sleeve has at least two or preferably exactly two protrusions or grooves as the coupling formation, and, namely for providing protection against tilting of the sleeve coupled to the coupling counter-formation, preferably on each side of the access opening, in each case at least one protrusion or at least one groove or particularly preferably in each case exactly one protrusion or exactly one groove. The design of grooves in side surfaces of the sleeve enables a design of the sleeve with at least two parallel surfaces with respective flat bevel surface, which have no overhang beyond the side surfaces, so that the sleeves or the breathing gas filter arrangements can be stacked in one direction orthogonally to these parallel flat side surfaces.

Alternatively, the coupling formation can include at least one protrusion, preferably two protrusions, wherein, again particularly preferably, in each case at least one protrusion is formed on each side of the access opening. The protrusion, which protrudes preferably in the direction away from the access opening from the skin of the sleeve, can give the preferably flexibly designed sleeve increased dimensional stability in the region of the access opening.

Independently of the design of the coupling formation as at least one groove or/and as at least one protrusion, for the advantageous sealing of the sleeve with respect to the filter heater or the filter carrier, in the transfer state, the at least one groove or/and the at least one protrusion extends along the coupling path over the entire length of the access opening.

Usually, the breathing gas filters are planar structures, that is to say objects which have a substantially larger dimension in two directions in space which are orthogonal to one another than in a thickness direction orthogonal to each of the two orthogonal directions in space. For the efficient use of space, the sleeve preferably has a shape which matches the breathing gas filter, so that the sleeve also has a substantially larger dimension in two directions in space which are orthogonal to one another than in a thickness direction orthogonal to each of the two directions in space which are orthogonal to one another.

To avoid incorrect arrangements, preferably both the sleeve and also the breathing gas filter accommodated therein are mirror symmetrical with respect to a mirror symmetry plane orthogonal to the thickness direction or shape invariant under a rotation by 180° with respect to an invariance axis orthogonal to the thickness direction or to the access opening surface Thus, preferably, a throughflow direction of the breathing gas filter is not crucial. Preferably, there can be throughflow in each of two possible opposite throughflow directions.

A particular advantage of the introduction of a breathing gas filter into the filter receiving space from a sleeve consists of the possibility of sealing the sleeve at least in the transfer state with respect to the filter carrier or with respect to the filter heater, so that during the filter replacement a breathing gas pressure prevailing in the respiratory line can be largely or even completely maintained. This applies particularly to the positive end-expiratory pressure (PEEP) which is important during ventilation. Therefore, according to an advantageous development of the present respiratory line or/and of the breathing gas filter arrangement, it is provided that at least one formation, of coupling formation and coupling counter-formation, has a sealing structure which at least in the transfer state sealingly lies against the respective other structure.

However, it should be pointed out here that, without a separate sealing structure, gaps present between coupling formation and coupling counter-formation can also achieve sufficient sealing and thus maintenance of a breathing gas pressure in the respiratory line for the duration of the filter replacement. This applies particularly if such a gap, like a labyrinth seal, is multiply angled in its course from a first space to a second space to be separated from the first space.

Preferably, the sealing structure is formed or arranged on the coupling counter-formation, since the coupling counter-formation can then be formed on a more dimensionally stable object such as, for example, a housing of a filter heater or on the filter carrier, and thus a sealing structure carried by the coupling counter-formation can be supported by an advantageously stiff and stable substrate. Preferably, the sealing structure is a sealing strip, particularly preferably with a sealing lip protruding away from the substrate supporting the sealing structure. The sealing strip preferably extends along the coupling path. In order to seal off the sleeve with the access opening as effectively as possible with respect to the filter heater with the passage opening or with respect to the filter carrier with the introduction opening in the transfer state, in each case at least one sealing strip runs on the filter heater or on the filter carrier on both sides of the passage opening or of the introduction opening, and namely over a length which corresponds at least to the length of the access opening along the coupling path, is preferably greater than the length of the access opening along the coupling path. For reasons relating to the most effective possible sealing, each of the sealing strips is preferably longer than the longer, along the coupling path, of the two openings facing one another in the transfer state, namely access opening and passage opening or access opening and introduction opening.

Preferably, the sealing structure is produced from a less stiff material than the formation, of coupling formation and coupling counter-formation, supporting it. A deformation formation on a groove is also considered as sealing structure, wherein the deformation formation is designed in such a manner that it lies against a protrusion introduced into the groove in a protrusion deforming and thus sealing manner. This applies particularly to the case in which the protrusion is made of an elastomer which is in principle also suitable for sealing tasks, such as, for example, rubber or silicone. The sealing structure designed as deformation formation can then be made of the same material or even of a stiffer material than the formation supporting it. This naturally also applies mutatis mutandis to a sealing structure designed as deformation formation on the protrusion, which is used to deform a section of the groove.

A deformation formation as sealing structure can be designed so that it tapers in the direction of the formation supporting it, in order to facilitate, by higher surface compression on its far end which is at a distance from the formation, a deformation of the component section lying against it. Preferably, the sealing structure, and this also applies to the deformation formation, along the coupling path, at least in a region in which the sealing structure runs along the coupling path next to one of the facing openings in the transfer state, has a constant cross section at least in sections, preferably in its entirety.

The aforementioned advantages are described above on the basis of the introduction of a new breathing gas filter into the filter receiving space. However, they also relate to the removal of a used-up breathing gas filter from the filter receiving space. Therefore, it is preferably provided that the breathing gas filter can be removed along a removal movement path from the filter receiving space, wherein the filter carrier or the filter heater has an additional coupling counter-formation, wherein a sleeve can be coupled to the additional coupling counter-formation in such a manner that, in a delivery state, in which the access opening of the sleeve is aligned with the removal opening along the removal movement path, the sleeve is held on the additional coupling counter-formation. Preferably, the removal movement path is the aforementioned guide path defined by the guiding structure.

An empty sleeve which remains after the introduction of the breathing gas filter from the sleeve into the filter receiving space can thus be kept and, as reception sleeve during the next filter replacement, before the displacement out of the used breathing gas filter by the new breathing gas filter, can be coupled by its coupling formation to the additional coupling counter-formation. Then, the used breathing gas filter inaccessible to the personnel performing the filter replacement can be shifted into the reception sleeve.

The additional coupling counter-formation is preferably designed like the above-described coupling counter-formation, so that it can cooperate with the coupling formation in the sleeve in the same way as the coupling counter-formation. The above-described developments of the coupling counter-formation are therefore also developments of the additional coupling counter-formation.

The introduction of a new breathing gas filter from a breathing gas filter arrangement in coupling engagement with a coupling counter-formation and a resulting displacement of a used-up breathing gas filter out of the operating position into a reception sleeve in coupling engagement with the additional coupling counter-formation enable a particularly advantageous lasting maintenance of the breathing gas pressure prevailing in the respiratory line. As a result, the filter replacement can be carried out in the case of ongoing artificial respiration, almost without taking into consideration the respiratory situation of a patient connected to the respiratory line.

As already described above, the breathing gas filter preferably has a gas impermeable frame surrounding a filter material. This frame can reliably support a centrally accessible filter material accommodated in the frame and hold it in the breathing gas flow. An advantageously efficient and lasting throughflow of breathing gas through the filter material, while avoiding as much as possible leaks flowing out of the filter receiving space, can be achieved in that, spaced from the breathing gas flow path, preferably between the breathing gas flow path and the introduction opening or/and the removal opening, more preferably at the edge region of the introduction opening or/and of the removal opening, a sealing formation, in particular a sealing lip, is provided, which sealing lip in the operating position of the breathing gas filter lies sealingly against the frame of the breathing gas filter, preferably between the breathing gas flow path and the introduction opening or/and the removal opening, more preferably at the edge region of the introduction opening or/and of the removal opening. In order to achieve the most comprehensive possible and long-lasting sealing action, the sealing formation runs preferably runs in a closed path around the breathing gas flow path, wherein the portion of the filter material of the breathing gas filter accessible to the breathing gas flow, is preferably entirely located radially within the region enclosed by the sealing formation.

In principle, this sealing formation can also be arranged or/and formed on the frame of the breathing gas filter. However, the smoothest possible boundary surface of the frame orthogonal to the throughflow direction of the filter material is preferable for simplified stacking also of larger quantities of breathing gas filters or breathing gas filter arrangements.

The present invention also relates to a breathing gas filter arrangement as already described above, including a breathing gas filter accommodated in a sleeve, wherein the sleeve has a coupling formation for coupling with a coupling counter-formation of a filter carrier or of a filter heater as well as an access opening for the delivery of the breathing gas filter from the sleeve. Preferably, a wall section of the sleeve can be displaced relative to the coupling formation in such a manner that the breathing gas filter, as a result of displacement of the displaceable wall section relative to the coupling formation, can be shifted through the access opening out of the sleeve. Developments and embodiments of the sleeve or/and of the breathing gas filter which have already been described above are developments of the breathing gas filter arrangement.

So that the breathing gas filter can be stored in a sterile manner in the sleeve until delivery through the access opening, the access opening, in the storage state before coupling to the filter carrier or to the filter heater, is preferably closed by a removable or/and destructible closure. The closure can be formed by a material strip which covers the access opening and which can be removed from the access opening, in particular pulled off. The closure can be formed by a separately designed cover, the cover surface of which covers the access opening in the storage state, and a collar projects from its cover surface, which, in the storage state, protrudes into an interior space surrounded by the sleeve and there lies against inner surface sections of the sleeve.

The closure can also be formed by a material strip which, by a shifting of the breathing gas filter out of the sleeve, can be destroyed, for example, broken or torn. The closure can have, as rupturing closure, a predetermined breaking point designed as material weakening, where the closure loses its material cohesion when a limit load exerted by the breathing gas filter pressed against the closure is exceeded. The material weakening can be a perforation or a thin spot in the material, for example, due to embossing or partial cutting over less than the entire material thickness and the like.

A plurality of breathing gas filter arrangements can be accommodated stacked in a dispenser container. The dispenser container can have a dispenser opening through which in each case a breathing gas filter arrangement of the stack can be removed. Preferably, the dispenser opening is designed, in the state of use of the dispenser container, at a geodetically lower end so that, after removal of a breathing gas filter arrangement through the dispenser opening, the rest of the stack remaining in the dispenser container moves downward driven by gravity and a breathing gas filter arrangement, ready for removal, in the dispenser container is again made available for removal in front of the opening.

The present invention also relates to a respiratory device for at least partial artificial respiration of a living patient, including:
a breathing gas source,
a respiratory line arrangement, for conveying inspiratory breathing gas from the breathing gas source to a patient-side proximal breathing gas outlet opening and for conveying convey expiratory breathing gas away from a proximal breathing gas inlet opening,
a flow sensor arrangement for quantitative acquisition of the inspiratory or/and expiratory breathing gas flow in the respiratory line arrangement,
a pressure change device for changing the pressure of the breathing gas in the respiratory line arrangement as well as
a control device for operating the breathing gas source or/and the pressure change device,
wherein the respiratory line arrangement has a respiratory line designed and developed as described above.

The breathing gas source can be a fan which collects ambient air from the environmental atmosphere, it can be a storage container with breathing gas or it can be a connection formation for connection to a building installation for breathing gas supply as commonly found in clinics.

The pressure change device can also be the fan which is also part of the breathing gas source. Additionally or alternatively, the pressure change device can include at least one valve.

The present invention is described below in greater detail in reference to the attached drawings. In the drawings:

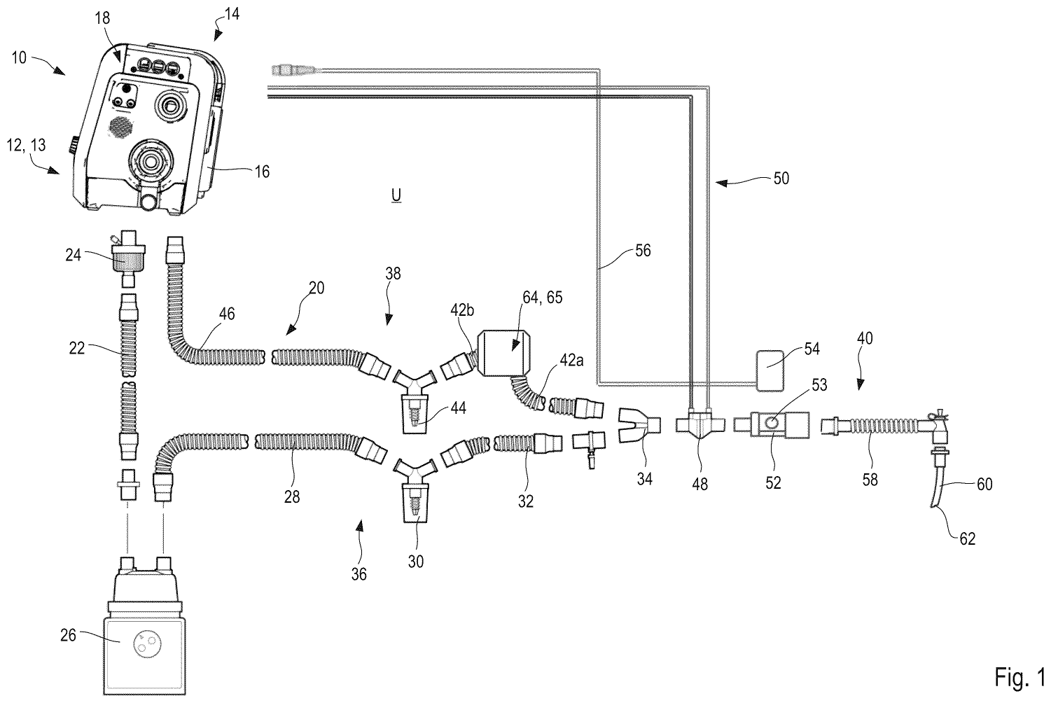

FIG. 1 represents an exploded view of a respiratory device according to the invention,

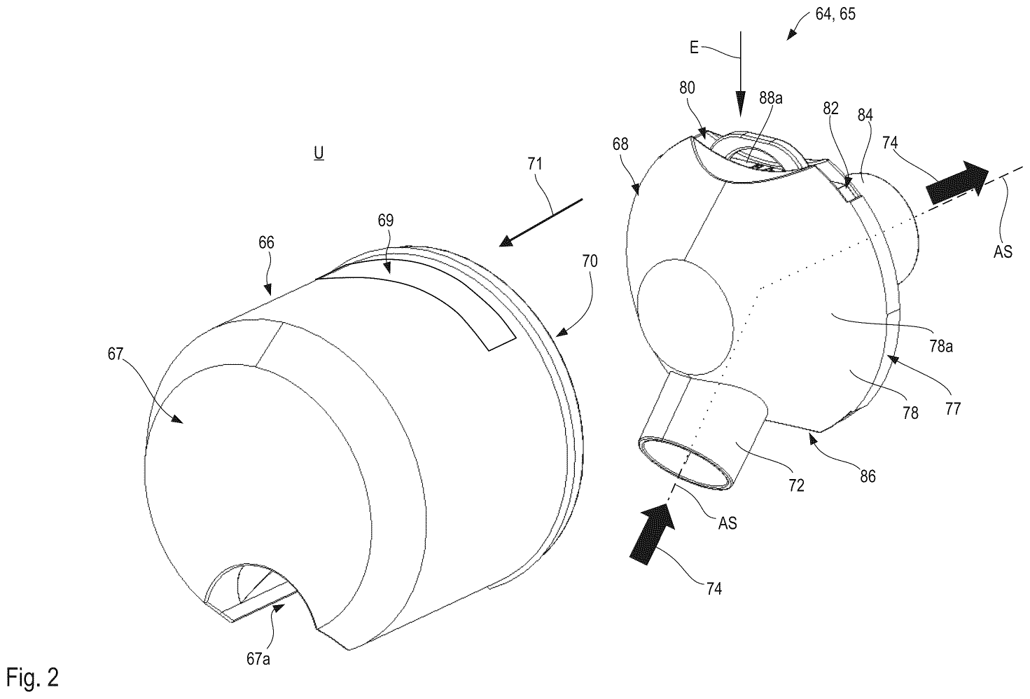

FIG. 2 represents a rough diagrammatic exploded view of a first embodiment of a respiratory line according to the invention as component of the respiratory device of FIG. 1,

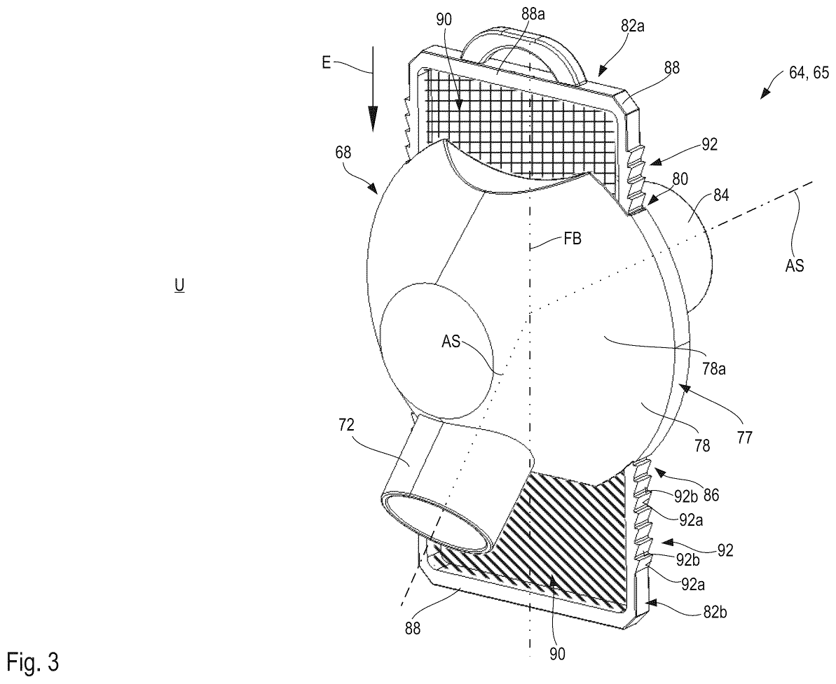

FIG. 3 represents a rough diagrammatic perspective view of a breathing gas filter change (filter replacement) on the respiratory line of FIG. 2,

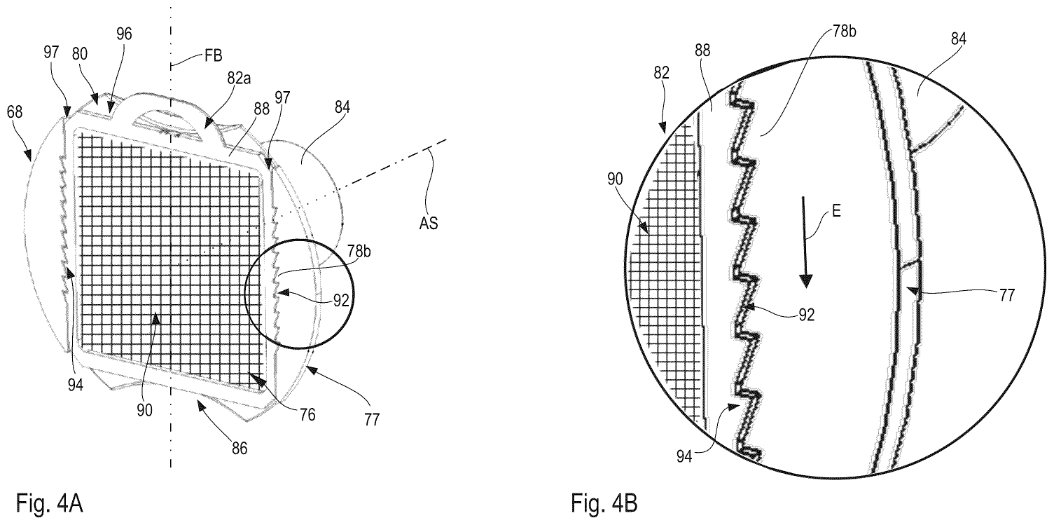

FIG. 4A represents a rough diagrammatic cross-sectional view of the filter carrier of FIGS. 2 and 3 with breathing gas filter arranged therein in the operating position,

FIG. 4B represents an enlarged view of the circled detail of FIG. 4A,

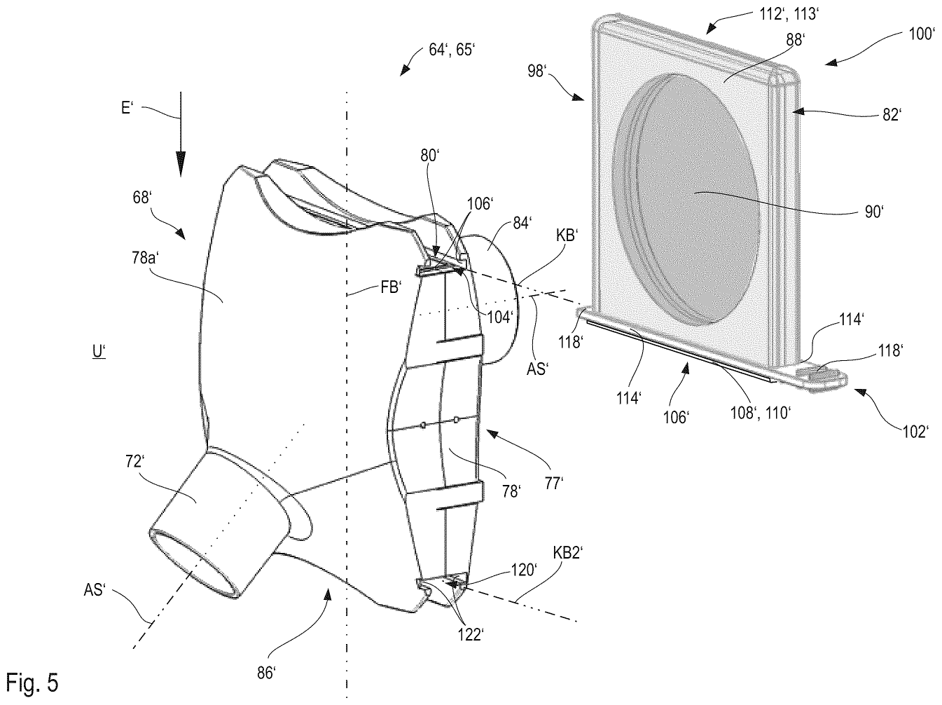

FIG. 5 represents a rough diagrammatic perspective view of a second embodiment of a respiratory line according to the invention as component of the respiratory device of FIG. 1,

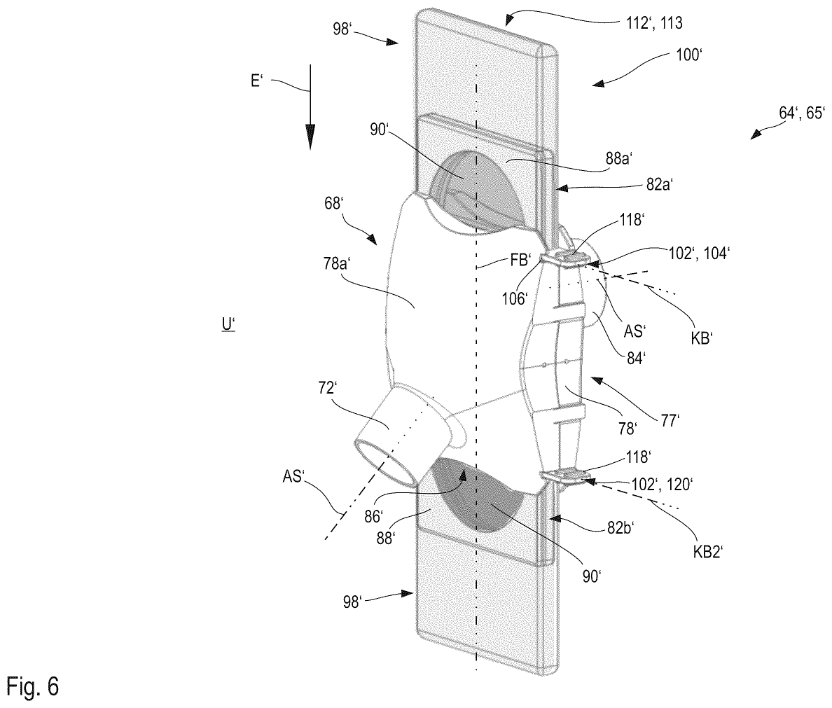

FIG. 6 represents a rough diagrammatic perspective view of the respiratory line of FIG. 5 during a filter replacement,

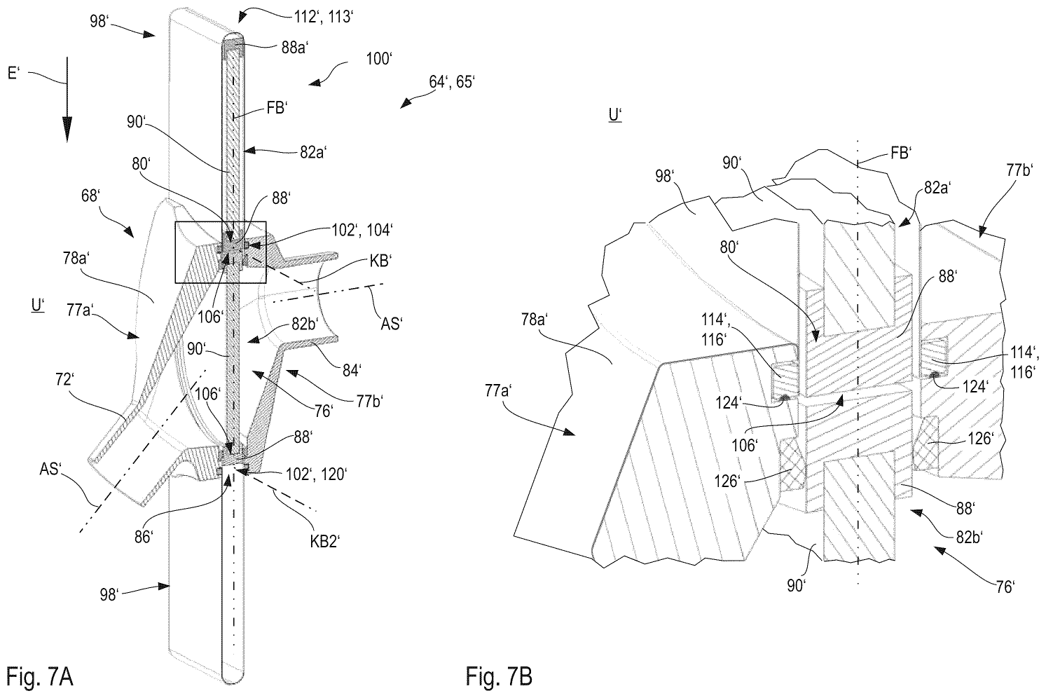

FIG. 7A represents a rough diagrammatic perspective view in longitudinal section of the respiratory line of FIGS. 5 and 6 before a filter replacement,

FIG. 7B represents an enlarged view of the marked rectangular region of FIG. 7A,

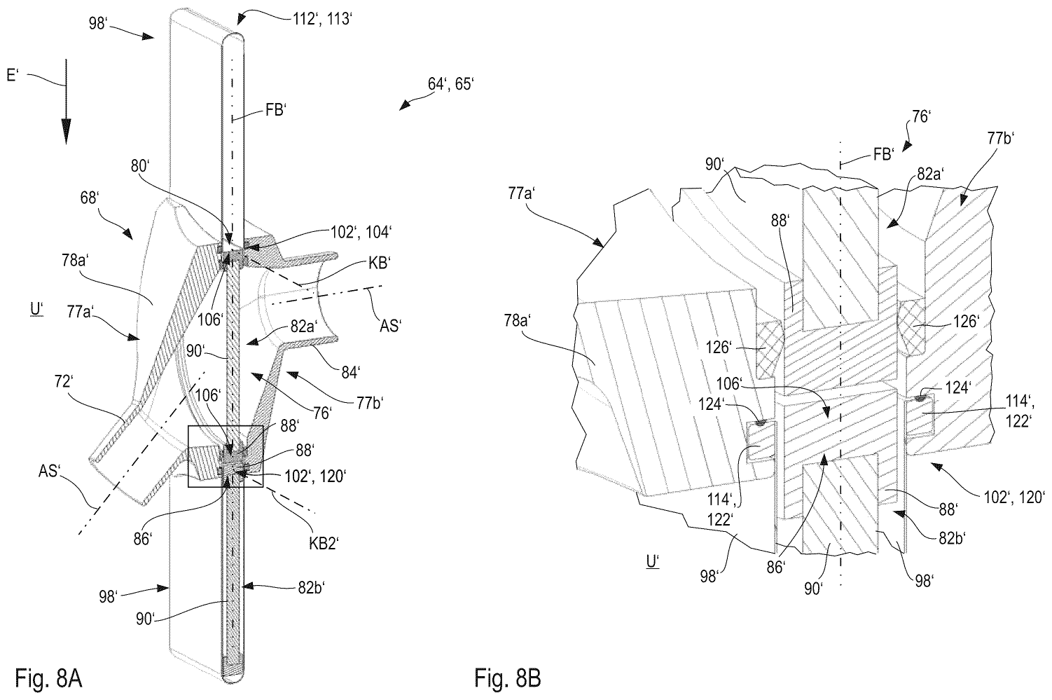

FIG. 8A represents a rough diagrammatic perspective view in longitudinal section of the respiratory line of FIGS. 5 and 6 after a filter replacement,

FIG. 8B represents an enlarged view of the marked rectangular region of FIG. 8A, and

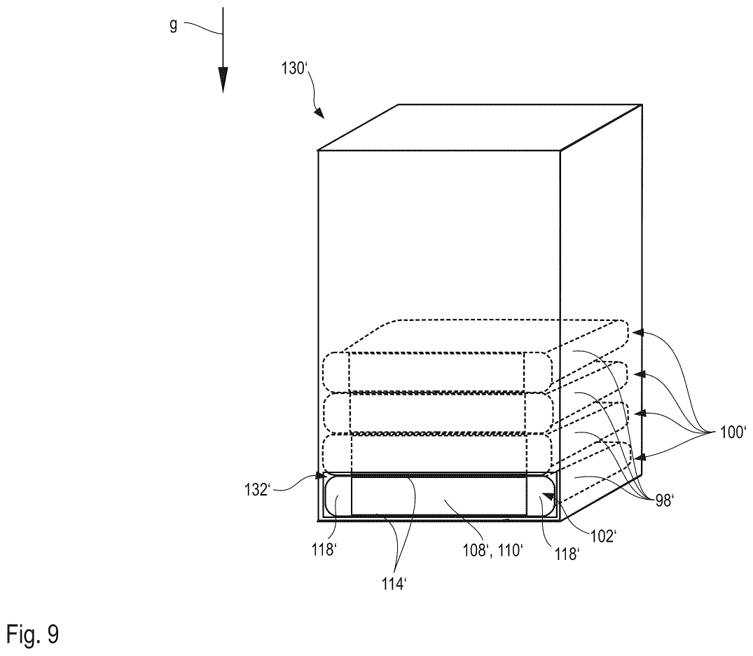

FIG. 9 represents a rough diagrammatic perspective view of a dispenser container for individual delivery of breathing gas filter arrangements of the present invention.

In FIG. 1, an embodiment according to the invention of a respiratory device as a whole is designated by 10. The respiratory device 10 includes a breathing gas source 12 in the form of a fan as well as a control device 14 for setting operating parameters of the breathing gas source 12. The breathing gas source 12 and the control device 14 are accommodated in the same housing 16. In this housing, valves known per se are also located, such as an inspiration valve and an expiration valve. However, they are not represented specifically in FIG. 1.

The control device 14 of the respiratory device 10 has an input/output device 18 which includes numerous switches, such as push-button switches and rotary switches, in order to be able to enter data into the control device 14 if needed. The fan of the breathing gas source 12 can be varied in terms of its delivery rate by the control device 14, in order to change the quantity of breathing gas delivered by the breathing gas source per unit of time. In the present embodiment example, the breathing gas source 12 is therefore also a pressure change device 13 of the respiratory device 10.

To the breathing gas source 12, a respiratory line arrangement 20 is connected, which includes seven flexible hoses in the present example. A first inspiratory breathing hose 22 runs from an optional filter 24 arranged between the breathing gas source 12 and itself to a conditioning device 26, where the breathing gas supplied by the breathing gas source 12 is moistened to a predetermined degree of humidity and optionally provided with aerosol drugs. The filter 24 filters and cleans the environmental air supplied by the fan as the breathing gas source 12.

A second inspiratory breathing hose 28 leads from the conditioning device 26 to an inspiratory water trap 30. A third inspiratory breathing hose 32 leads from the water trap 30 to a Y connector 34 which connects the distal inspiration line 36 and the distal expiration line 38 to a combined proximal inspiratory-expiratory respiratory line 40.

From the Y connector 34 back to the housing 16, a first expiratory breathing hose 42a runs to a breathing gas filter device 64. From said breathing gas filter device, a second expiratory breathing hose 42b runs to an expiratory water trap 44, and from there a third expiratory breathing hose 46 runs to the housing 16, where the expiratory breathing gas is released via an expiration valve, not shown, into the environment U.

On the combined inspiratory-expiratory side of the Y connector 34 near the patient, directly following the Y connector 34, a throughflow sensor 48 is arranged, here: a differential pressure throughflow sensor 48 which acquires the inspiratory and expiratory flows of breathing gas to the patient and away from the patient. A line arrangement 50 transmits the gas pressure prevailing on both sides of a flow barrier in the throughflow sensor 48 to the control device 14 which, from the transmitted gas pressures and in particular from the difference of the gas pressures, calculates the quantity of inspiratory and expiratory breathing gas flowing per unit of time.

In the direction away from the Y connector 34, toward the patient, following the throughflow sensor 48 is a measurement cuvette 52 for non-dispersive infrared acquisition of a predetermined gas proportion in the breathing gas. In the represented example, the proportion of CO₂ in the breathing gas is to be determined. Here, the CO₂ proportion preferably both in the inspiratory breathing gas and the expiratory breathing gas is of interest, since the change of the CO₂ proportion between inspiration and expiration is a measure of the metabolic capacity of the patient's lung.

The sensor module 54 can be detachably coupled to the measurement cuvette 52 in such a manner that the sensor module 54 can irradiate the measurement cuvette 52 with infrared light through window 53. From the spectral intensity of the infrared light, after irradiation of the measurement cuvette 52, on the basis of the degree of absorption of infrared light, a conclusion can be drawn in a known manner as to the quantity or the proportion of CO₂ in the breathing gas.

The sensor module 54 is connected via a data line 56 to the control device 14 of the respiratory device 10 and transmits, via the data line 56, intensity information of the acquired infrared light to the control device 14 for evaluation.

The measurement cuvette 52 is followed in the direction toward the patient by an additional hose piece 58, on which an endotracheal tube 60 is arranged as breathing interface to the patient. A proximal opening 62 of the endotracheal tube 60 is both a breathing gas outlet opening through which inspiratory breathing gas is conveyed through the endotracheal tube 60 into the patient and also a breathing gas inlet opening through which expiratory breathing gas is conveyed from the patient back into the endotracheal tube 60.

Designated by reference numeral 64 is a heatable breathing gas filter device arranged in the first expiratory breathing hose 42 which will be described in greater detail below in reference to FIGS. 2 to 4.

As explained below in reference to FIG. 2, the heatable breathing gas filter device 64 forms an embodiment of a respiratory line 65 of the present invention.

In FIG. 2, the breathing gas filter device 64 is represented in a rough diagrammatic exploded view. A filter heater 66 and a filter carrier 68 which can be detachably inserted into the filter heater 66 can be seen.

On the side facing away from the observer of FIG. 2, the filter heater 66 includes a filter carrier accommodation 70, into which the filter carrier 68 can be inserted along the arrow 71.

The housing 67 of the filter heater 66 has a recess 67a on its lower side, which is used for accommodating a feed-side connection nozzle 72 of the filter carrier 68, when the filter carrier 68 is accommodated in its operating position in the filter carrier accommodation 70 of the filter heater 66.

The connection nozzle 72 is part of the breathing gas flow conveying of the respiratory line 65. In the connection nozzle 72, breathing gas marked by thick arrows 74 in its flow direction, in the present case expiratory breathing gas, is conveyed into the filter carrier 68, more precisely into its filter receiving space 76 visible in FIG. 4A in the filter housing 77. The connection nozzle 72 can be used for fluid-flow-mechanical connection of the first expiratory breathing hose 42a. Here, the breathing gas 74 flows along a virtual breathing gas flow path AS, which is conceived as centrally passing through the connection nozzle 72, which is of cylindrical design, for example, and which, in the example of the cylindrical connection nozzle 72, coincides with its cylinder axis. In the interior of the filter carrier 68, the further course of the virtual breathing gas flow path AS that is covered by the wall 78 of the filter carrier 68 is indicated by a dotted line.

With respect to the virtual breathing gas flow path AS, with a radial spacing from said path, the wall 78 of the filter carrier 68 has an inlet opening 80, through which a breathing gas filter 82 can be shifted into the filter carrier 68 along the introduction direction E. The breathing gas filter 82 in FIG. 2 is in its operating position, in which it cleans the breathing gas 74 flowing through the filter carrier 68.

The wall 78 of the filter carrier 68, in the represented example, has a frustoconical section 78a. However, this section can have any other shape. The frustoconical section 78a offers the advantage of a more reliable large-area coupling to the filter heater 66, in order to be able to evenly transfer a sufficient quantity of heat per unit of time via the surface of the frustoconical section 78a.

After passage through the filter receiving space 76 and the breathing gas filter 82 optionally accommodated therein, the breathing gas 74 flows through the outlet-side connection nozzle 84 out of the filter carrier 68.

The filter carrier 68, on its outlet side, in turn can have a conical section which tapers starting from the filter receiving space 76, wherein the outlet-side connection nozzle 84 which in turn can be used for fluid mechanical connection of a breathing hose, for example, of the second expiratory breathing hose 42b, is arranged approximately centrally on the outlet side of the filter carrier 68. Alternatively, the outlet-side connection nozzle 84 can be arranged off center on the outlet side of the filter carrier 68, if this is advantageous for the further line course.

As can be readily seen in FIG. 2, the feed-side section of the breathing gas flow path AS in the region of the feed-side connection nozzle 72 and of the outlet-side section of the breathing gas flow path AS in the region of the outlet-side connection nozzle 84 are angled with respect to one another, so that, in spite of the feed-side connection nozzle 72 which projects out from the filter carrier 68, it is possible to put the largest possible surface area of the filter carrier 68 contiguously surrounded by the filter heater 66 in a heat transfer relationship with said filter heater.

Particularly advantageously, the wall 67 of the filter heater 66 has a passage opening 69 which, when the filter carrier 68 is located in the filter heater 66 in its operational position, is aligned with the introduction opening 80 of the filter carrier 68 along the introduction direction E. As a result, it is possible to introduce a breathing gas filter 82 through the passage opening 69 and the introduction opening 80 into the filter carrier 68 without for this purpose having to remove the filter carrier 68 from the filter heater 66. The filter heater 66 can be removed from the filter carrier 68 without the respiratory line having to be opened or/and the breathing gas flow having to be interrupted for this purpose.

The arrangement of the introduction opening 80 such that it is formed in a wall 78 of the filter carrier 68 located at distance radially from the breathing gas flow path AS enables the introduction of a breathing gas filter 82 into the filter receiving space 76 without the flow path of the breathing gas 74 and thus the artificial respiration of the patient having to be interrupted for this purpose.

Shown in FIG. 3 is a filter replacement which is particularly advantageous since it is both simpler and more hygienic. By introduction of a new breathing gas filter 82a through the introduction opening 80 in introduction direction E, a used-up breathing gas filter 82b is shifted out of a facing removal opening 86 which, with respect to the introduction direction E, is opposite the introduction opening 80 on the wall 78 of the filter carrier 68. The used-up breathing gas filter 82b consequently does not have to be touched for removal from the filter carrier 68. For example, if a collection container is arranged under the removal opening 86, then the used-up breathing gas filter 82b can be simply pressed or shifted out by the new breathing gas filter 82a into the collection container. Subsequently, the used-up breathing gas filter 82b can be disposed of, again without needing to be touched by operating personnel.

The breathing gas filters 82 have a filter frame 88 which, in the represented example, circumferentially surrounds a flat filter material 90. Thereby, sufficient force can be exerted by the new breathing gas filter 82a to be introduced into the filter carrier 68 on the used-up breathing gas filter 82b to be removed from out of the filter carrier 68.

The filter frames 88 are made of gas impermeable material, so that in FIG. 2 a frame section 88a protruding in the operating position of the breathing gas filter 82 out of the introduction opening 80 can seal off the introduction opening 80 and an opposite frame section can also seal off the removal opening 86. Thus, simultaneously with the introduction of a breathing gas filter 82 into its operating position in the filter carrier 68, the filter receiving space 76 accommodating the breathing gas filter 82 is sealed off by the breathing gas filter 82, more precisely by its frame 88, with respect to the outside environment U. At the removal opening 86, a section of the frame 88 of the breathing gas filter 82 introduced into the operating position can protrude from the removal opening 86. However, this does not have to be the case.

On the frame sides of the filter frames 88, an inhibiting counter-formation 92 designed as a toothing, in particular a sawtooth toothing with successive differently inclined tooth surfaces 92 and 92b, is provided, which cooperates with a complementarily designed inhibiting formation 94 of the filter carrier 68, which can be seen particularly well in FIG. 4B, in order to allow only a movement of a breathing gas filter 82 in introduction direction E through the introduction opening 80 relative to the filter carrier 68 and in order to inhibit a movement of the breathing gas filter 82 in the opposite direction.

A breathing gas filter 82 can thus be introduced into the filter carrier 68 or into the filter receiving space 76 and be removed therefrom along a preferred straight bend-free guide path FB. The breathing gas filter 82 can thus be moved along the straight bend-free guide path FB through the filter carrier 68.

As can be seen in FIGS. 4A and 4B, formed on a wall section 78b of the wall 78 of the filter carrier 68, which laterally delimits the filter receiving space 76, is an inhibiting formation 94 which is complementary to the inhibiting counter-formation 92 and which cooperates with the inhibiting counter-formation 92 in positive-locking connection in such a manner that the breathing gas filter 82 can be moved only unidirectionally along the guide path FB through the filter carrier 68. The inhibiting counter-formation 92 and the inhibiting formation 94 do not have to be designed as strictly complementary to one another. It is sufficient if the desired inhibiting action is achieved.

The introduction opening 80 and the removal opening 86 are end regions of a channel 96 which passes through the filter carrier 68 along the guide path FB and which forms the filter receiving space 76. The side limits of the channel 96 through the filter carrier 68 or its wall 78 form a guiding structure 97 which, as a result of its dimensions, allows an only unidirectional translational, that is to say rotation-free, movement of the breathing gas filter 82 through the introduction opening 80 into the operating position and out of the operating position through the removal opening 86. Thus, the breathing gas filter 82, in its operating position, is accommodated advantageously without play and with formation of a gas seal on the introduction opening 80 and the removal opening 86 in the filter carrier 68.

The filter 24, in contrast to the representation in FIG. 1, can be omitted or it can be designed like the breathing gas filter device 64 or the respiratory line 65.

The arrangement site of the breathing gas filter device 64 in the respiratory line arrangement 20 in the embodiment example is only an example. Alternatively to the representation of FIG. 1, the breathing gas filter device 64 can also be arranged between the water trap 44 and the expiration valve.

Represented in FIGS. 5 to 8B is a second embodiment of a respiratory line 65′ with a breathing gas filter device 64′. Identical and functionally identical components and component sections are provided in the second embodiment with the same reference numerals as in the first embodiment of FIGS. 1 to 4B but with the addition of an apostrophe. The second embodiment is described below only to the extent that it differs from the above-described first embodiment, and otherwise reference is explicitly made to the description of the first embodiment for the explanation of the second embodiment.

The respiratory line 65′ of the second embodiment with its breathing gas filter device 64′ is represented without filter heater simply for reasons of clarity. The breathing gas conveyance device 64′ of the second embodiment can also have a filter heater.

The breathing gas filter 82′ is represented in FIG. 5 in its storage state, in which it is accommodated in a sleeve 98′. The sleeve 98′ and the breathing gas filter 82′ accommodated therein form a breathing gas filter arrangement 100′.

The sleeve 98′ and thus the breathing gas filter arrangement 100′ as a whole have a coupling formation 102′ which is used for establishing a coupling engagement with a coupling counter-formation 104′ on the housing 77′ of the filter carrier 68′. In the represented example, the sleeve 98′ itself is produced from an elastomer material, for example, from rubber or from silicone, and it surrounds the breathing gas filter 82′ with small clearance or lies directly against the frame 88′ of the breathing gas filter 82′.

The breathing gas filter 82′ is represented without inhibiting formation but it usually has such an inhibiting formation.

The coupling formation 102′ surrounds an access opening 106′, not visible in FIG. 5, through which the breathing gas filter 82′ can be discharged from the sleeve 98′. The access opening 106′ is closed by a closure 108′ in the form of a film 110′ which is affixed as seal onto the coupling formation 102′ and which can be opened in that the breathing gas filter 82′ is pressed through the film 110′ which is provided with a predetermined breaking point. For this purpose, on the longitudinal end 112′ of the elastomer sleeve 98′ opposite the coupling formation 102′, at least one wall section 113′ can be shifted in a deforming manner toward the coupling formation 102′.

The coupling formation 102′ includes on both sides of the access opening 106′ in each case a protrusion 114′ continuously running over the entire extension length of the sleeve 98′ and beyond, which protrusion preferably protrudes with respect to the main extension plane of the sleeve 98′. The two protrusions 114′ can be shifted along the coupling path KB′ orthogonal to the guide path FB′ in each case into one groove 116′ of the coupling counter-formation 104′. Handle sections 118′ on the two longitudinal ends of the coupling formation 102′ facilitate handling of the sleeve 98′ or of the breathing gas filter arrangement 100′ during the introduction of the protrusions 114′ into the grooves 116′ and during the displacement of the sleeve 98′ along the coupling path KB′.

The two grooves 116′ extend on both sides of the introduction opening 80′ along the coupling path KB′ over the entire length of the introduction opening 80′ and beyond on both sides of the introduction opening 80′.

In contrast to the first embodiment, the frame 88′ of the breathing gas filter 82′ has, for example, a circular opening, through which the flat filter material 90′ is accessible to the breathing gas flow for throughflow. Naturally, the opening in the frame 88′ can also be rectangular or in general polygonal.

In FIG. 6, a filter replacement of breathing gas filters 82′ of the second embodiment is shown. The representation of FIG. 6 therefore corresponds to the representation of FIG. 3, except for that it uses the second embodiment of the breathing gas filter 82′.

After the establishment of a coupling engagement between the coupling formation 102′ and the coupling counter-formation 104′, more precisely between the protrusions 114′ and the associated grooves 116′, the breathing gas filter arrangement 100′ has been brought into the transfer state, in which the access opening 106′ and the introduction opening 80′ are aligned with one another along the guide path FB′ which in the present case represents an introduction movement path.

In the designated transfer state, due to the pressure on the wall section 113′ on the longitudinal end 112′ of the sleeve 98′ or of the breathing gas filter arrangement 100′, the longitudinal end 112′ has been shifted toward the access opening 106′ with deformation of the sleeve 98′, and, as a result, the closure 108′ is broken. As a result of the thus opened access opening 106′, a new breathing gas filter 82a′ is introduced along the guide path FB′ into the filter receiving space of the filter carrier 68′, and in the process simultaneously an old or used-up breathing gas filter 82b′ is shifted along the same guide path FB′ out of the filter receiving space through the removal opening 86′. The guide path FB′ is therefore also a removal movement path. Likewise, the introduction direction E or E′ is also the removal direction E or E′.

As an aforementioned collection container, before the beginning of the filter replacement, an additional sleeve 98′ which is identical to the above-described sleeve 98′ has been arranged on the filter carrier 68′. For this purpose, the filter carrier 68′ has an additional coupling counter-formation 120′ which is designed identically to the aforementioned coupling counter-formation 104′. The additional coupling counter-formation 120′ is simply arranged on the other side of the breathing gas flow path AS' with respect to the coupling counter-formation 104′. What was said above about the coupling counter-formation 104′ thus also applies to the additional coupling counter-formation 120′.

The additional sleeve 98′ has been brought with its coupling formation 102′, more precisely with its protrusions 114′, in coupling engagement with the additional coupling counter-formation 120′, more precisely with its grooves 122′. The additional sleeve 98′ is in a delivery state in FIG. 6, in which the access opening of the additional sleeve 98′ is aligned with the removal opening 86′ along the guide path FB′. Thus, the used-up breathing gas filter 82b′ is dislodged by introducing the new breathing gas filter 82a′ into the additional sleeve 98′ and, after the filter replacement has occurred, it can be removed from the filter carrier 68′ and disposed of, surrounded by the additional sleeve 98′, without contact with a person, and disposed of.

By the coupling engagements of the two sleeves 98′ with the coupling counter-formations 104′ and 120′, the introduction opening 80′ and the removal opening 86′ are largely sealed with respect to the outside environment U′ during the filter replacement, so that a breathing gas pressure prevailing in the respiratory line 65′ is advantageously not affected by the filter replacement. Thus, a PEEP can also be maintained during the filter replacement.

A particularly advantageous sealing situation in the vicinity of both the introduction opening 80′ and the removal opening 86′ is explained below in reference to FIGS. 7A to 8B.

In FIG. 7A, a breathing gas filter device 64′ ready for the filter replacement is represented in longitudinal section. The breathing gas filter arrangement 100′ is in the transfer state, so that the new breathing gas filter 82a′ is ready for the filter replacement. The closing film 110′ has been removed in the case of FIG. 7A before the establishment of the coupling engagement of the coupling formation 102′ with the coupling counter-formation 104′. In the operating position in the filter receiving space 76′, a used-up breathing gas filter 82b′ is arranged. In addition, an empty sleeve 98′ with its coupling formation 102′ is in coupling engagement with the additional coupling counter-formation 120′ and is in the delivery state, so that the empty sleeve 98′ is ready to receive the breathing gas filter 82b′.

In FIG. 7B, the rectangular section marked in FIG. 7A is represented enlarged, in order to represent the sealing situation on the breathing gas filter device 64′ before the filter replacement.

In FIG. 7B, it can be easily seen how the protrusions 114′ protruding on both sides of the access opening 106′ on the sleeve 98′ away from the access opening 106′ in each case engage in its associated groove 116′ on the coupling counter-formation 104′ of the filter carrier housing 77′. The engagement extends over the entire length of the groove 116′.

On the surface of each groove 116′, which points opposite the introduction direction E′, in each case a sealing structure in the form of a sealing strip 124′ is arranged, which runs along the coupling path KB′ and protrudes from the surface of the groove 116′ into the groove space enclosed by the groove 116′. Each sealing strip 124′ therefore lies, by its end side away from the groove surface supporting it, against a surface of the protrusion 114′ introduced into the groove 116′, facing the groove surface. The sealing strip 124′ thus seals off a gap existing between each protrusion 114′ and the associated groove 116′ in which it engages. Thus, during a filter replacement, there is no direct flow path through the groove 116′ between the filter receiving space 76′, in which a desired breathing gas pressure prevails, and the environment U′.

In contrast to the representation, the sealing strips can additionally or alternatively be arranged on any other wall of the groove 116′, that, in the transfer state, is faced by an outer wall section of a protrusion 114′.

The sealing strip 124′ can be, for example, a line of silicone applied along the coupling path KB′ onto the groove surface in question or it can be any other desired soft elastic sealing material.

In addition to the sealing structure in the form of the sealing strips 124′, on the two filter carrier housing components 77a′ and 77b′ of the filter carrier housing 77′ in each case a sealing formation 126′ is arranged, which preferably runs in a closed path around the breathing gas flow path AS′.

As long as the breathing gas filter 82′ is correctly located in the filter receiving space 76′ arranged in the operating position, the portion of the filter material 90′ which can be reached by the breathing gas flow through the filter carrier housing 77′ is shielded from the outside environment U′ by the sealing formations 126′ lying against the frame 88′ of the breathing gas filter 82′ on both sides of the filter material 90′. The sealing formations 126′ are formed identically on both sides of the breathing gas filter 82′, for example, by a closed circumferential elastomer strip.

When the used-up breathing gas filter 82b′ is dislodged by the new breathing gas filter 82a′ along the guide path FB′ through the removal opening 86′, in a time period of the removal movement of the used-up breathing gas filter 82b′, the frame 88′ in sections comes out of contact engagement with the sealing formations 126′, since the filter material 90′, which can be reached according to the intended use by the breathing gas flow, overlaps with the sealing formations 126′. In this case, the sealing formations 126′, in the region in which they face the uncovered portion of the filter material 90′, which can be reached by the breathing gas flow, no longer completely seal off the breathing gas flow from the outside environment. This then occurs by means of only the sealing strips 124′, which, as a result of their sealing effect with respect to the coupling formation 102′, continue to ensure maintenance of the breathing gas pressure prevailing in the respiratory line 65′.

The representation of FIG. 8A corresponds to the representation of FIG. 7A but at a time after the filter replacement. FIG. 8B in turn shows the rectangular region marked in FIG. 8A in an enlargement.

By a deforming displacement of the wall section 113′ on the longitudinal end 112′ of the upper sleeve 98′ in FIGS. 7A and 8A toward the access opening 106′, the new breathing gas filter 82a′ has been brought into the filter receiving space 76′ without coming in direct contact with the operator. With the introduction movement of the new breathing gas filter 82a′, as a result of the contact engagement of the two frames 88′ of the new breathing gas filter 82a′ and of the used-up breathing gas filter 82b′, the used-up breathing gas filter 82b′ has been shifted out into the lower sleeve 98′ in FIGS. 7A and 8A.

The used-up breathing gas filter 82b′ can be shifted with the lower sleeve 98′, without direct contact with the breathing gas filter 82b′, along the lower coupling path KB2′ relative to the filter carrier 68′ and thus be detached from the filter carrier 68′.

The additional coupling counter-formation 120′ is mirror symmetrical with respect to a mirror symmetry plane parallel to the two coupling paths KB′ and KB2′. Therefore, the respective grooves 122′ of the additional coupling counter-formation 120′ limiting surfaces which point in removal direction or in introduction direction E′ each have a sealing strip 124′ (see FIG. 8A). Due to the mentioned mirror symmetry, the description of the grooves 116′ also applies to the grooves 122′ taking into consideration the mirror symmetry.

The sleeves 98′ can be introduced from both sides of the filter carrier 68′ along the respective coupling paths KB′ and KB2′ into the coupling counter-formation 104′ and into the additional coupling counter-formation 120′.

Thus, when, in the two coupling counter-formations 104′ and 120′, in each case a sleeve 98′ is arranged, sealing strips 124′ completely or at least nearly completely seal off the filter receiving space 76′ against the outside environment U′.

In FIG. 8A, the section of the circumferential sealing formation 126′ located closer to the removal opening 86′ can be seen, which now sealingly lies against the frame 88′ of the new breathing gas filter 82a′ which has just been introduced.

With the breathing gas filter device 64′ of the second embodiment, as represented in FIGS. 5 to 8B, a replacement of breathing gas filters 82′ can be carried out with ongoing respiratory operation without affecting the breathing gas pressure prevailing in the respiratory line 65′.

In FIG. 9, an advantageous dispenser container 130′ is shown, which has a plurality of breathing gas filter arrangements stacked in 100′ along the effective direction of the gravitational force g.

Through a dispenser opening 132′ on the geodetically lower end, that is to say the lower end in the effective direction of the gravitational force g, in each case the lowermost breathing gas filter arrangement 100′ is pulled out of the dispenser container 130′, while the rest of the breathing gas filter arrangements in 100′ still remaining in the dispenser container 130′ move driven by the force of gravity, so that, after the removal of a breathing gas filter arrangement 100′, the breathing gas filter arrangement 100′ previously located directly above it is now located in front of the dispenser opening 132′ and made available for removal from the dispenser container 130′.

The width of the dispenser opening 132′ in FIG. 9 along the effective direction of the gravitational force g is slightly greater than the width of the coupling formation 102′ to be measured in the same direction, so that the breathing gas filter arrangements in 100′ can only be removed singly through the dispenser opening 132′ from the dispenser container 130′. With the removal of the lowermost breathing gas filter arrangement 100′ available at the front dispenser opening 132′, the breathing gas filter arrangement 100′ lying directly over it is pulled off on the wall section of the dispenser container 100′ located over the dispenser opening 132′.

1. A respiratory line for conveying inspiratory or/and expiratory breathing gas during at least partial artificial respiration of a patient, wherein the respiratory line defines an imaginary virtual breathing gas flow path which is conceived as extending centrally through it, along which flow path the respiratory line conveys the breathing gas, wherein the respiratory line has a filter carrier in which a filter receiving space which is passed through by the breathing gas flow path is formed, in which a breathing gas filter, through which the breathing gas can flow, is replaceably accommodated,
wherein the filter carrier has a wall surrounding the filter receiving space with radial spacing from the virtual breathing gas flow path with an introduction opening arranged with radial spacing from the virtual breathing gas flow path, through which the breathing gas filter can be introduced into the filter receiving space and be brought into its operational operating position.

2. The respiratory line according to claim 1,
wherein the filter carrier has a guiding structure which guides an introduction movement of the breathing gas filter from the introduction opening into the filter receiving space.

3. The respiratory line according to claim 1,
wherein the wall (of the filter carrier has a removal opening different from the introduction opening and arranged with radial spacing from the virtual breathing gas flow path through which removal opening a breathing gas filter arranged in the filter receiving space is removable from the filter receiving space.

4. The respiratory line according to claim 2,
wherein the guiding structure is arranged between the introduction opening and the removal opening, so that it also guides a removal movement of a breathing gas filter from the filter receiving space.

5. The respiratory line according to claim 4,
wherein the breathing gas filter is movable along a bend-free guide path defined by the guiding structure through the introduction opening, toward the virtual breathing gas flow path, into the filter receiving space and into the operating position as well as out of the operating position, away from the virtual breathing gas flow path, out of the filter receiving space through the removal opening.

6. The respiratory line according to claim 1,
wherein the filter carrier has an inhibiting formation which allows a movement of the breathing gas filter through the introduction opening into the filter receiving space and which inhibits an oppositely directed movement of the breathing gas filter through the introduction opening.

7. The respiratory line according to claim 6,
wherein the breathing gas filter has an inhibiting counter-formation which cooperates with the inhibiting formation of the filter carrier for achieving the unidirectional movability of the breathing gas filter.

8. The respiratory line according to claim 6,
wherein the inhibiting formation and the inhibiting counter-formation form a plurality of unidirectionally surmountable catches located one after the other along the allowed movement direction.

9. The respiratory line according to claim 1,
wherein the breathing gas filter has a gas impermeable shoulder, which, when the breathing gas filter is accommodated in operational state in the filter receiving space, forms a flow barrier against throughflow of breathing gas through the introduction opening.

10. The respiratory line according to claim 1,
wherein the wall is part of a housing of the filter carrier, which surrounds the filter receiving space in the operational state, wherein the housing, as dividable housing, has at least two housing portions which is movable apart and closer together.

11. The respiratory line according to claim 1,
wherein the filter carrier, on both sides of the filter receiving space, has flow-conveying line sections, of which respective associated sections of the virtual breathing gas flow path enclose an angle with one another.

12. The respiratory line according to claim 1,
wherein the respiratory line has a filter heater which surrounds the filter carrier in the operational state at least in sections.

13. The respiratory line according to claim 12,
wherein the filter heater has a filter carrier accommodation formed as a component separate from the filter carrier, into which filter carrier accommodation the filter carrier is insertable and from which an inserted filter carrier is removable.

14. The respiratory line according to claim 12,
wherein the filter heater has a wall with a passage opening, wherein the passage opening, in the operational state, is aligned with the introduction opening in such a manner that a breathing gas filter is introduceable through the passage opening into the introduction opening and thus into the filter carrier.

15. The respiratory line according to claim 1,
wherein the breathing gas filter is introduced along an introduction movement path into the filter receiving space, wherein the breathing gas filter, in a starting state before its introduction into the filter receiving space, is accommodated in a sleeve, wherein the sleeve has a coupling formation which is coupled to a coupling counter-formation of the filter carrier or of the filter heater in such a manner that, in a transfer state in which an access opening of the sleeve, which is designed for the delivery of the breathing gas filter out of the sleeve, is aligned with the introduction opening along the introduction movement path, the sleeve is held on the coupling counter-formation, wherein at least one wall section of the sleeve can be shifted relative to the coupling formation in such a manner that the breathing gas filter, as a result of displacement of the displaceable wall section relative to the coupling formation, is shifted through the access opening out of the sleeve.

16. The respiratory line according to claim 15,
wherein the sleeve is designed to be flexible, in such a manner that the displacement which shifts the breathing gas filter through the access opening out of the sleeve is a deformation of the displaceable wall section.

17. The respiratory line according to claim 15,
wherein the coupling formation and the coupling counter-formation enable a displacement of the sleeve relative to the filter carrier along a coupling path which runs transversely to the introduction movement path of the breathing gas filter into the filter carrier.

18. The respiratory line according to claim 17,
wherein one formation of coupling formation and coupling counter-formation, includes at least one protrusion, and in that the respective other formation, of coupling formation and coupling counter-formation, has at least one groove, wherein the at least one protrusion engages, at least in the transfer state, in the at least one groove.

19. The respiratory line according to claim 15,
wherein at least one formation out of coupling formation and coupling counter-formation has a sealing structure which, at least in the transfer state, sealingly lies against the respective other formation.

20. The respiratory line according to claim 15,
wherein the breathing gas filter is removable along a removal movement path from the filter receiving space, wherein the filter carrier or the filter heater has an additional coupling counter-formation, wherein a sleeve is coupled to the additional coupling counter-formation in such a manner that, in a delivery state, in which the access opening of the sleeve is aligned with the removal opening along the removal movement path, the sleeve is held on the additional coupling counter-formation.

21. The respiratory line according to claim 1,
wherein the breathing gas filter has a gas impermeable frame surrounding a filter material, wherein, with a spacing from the breathing gas flow path, a sealing formation is provided, which, in the operating position of the breathing gas filter, sealingly lies against the frame (88′) of the breathing gas filter.

22. A respiratory device for at least partial artificial respiration of a living patient, including:
a breathing gas source,
a respiratory line arrangement, in order to convey inspiratory breathing gas from the breathing gas source to a patient-side proximal breathing gas outlet opening and in order to convey expiratory breathing gas away from a proximal breathing gas inlet opening,
a flow sensor arrangement for quantitative acquisition of the inspiratory or/and of the expiratory breathing gas in the respiratory line arrangement,
a pressure change device for changing the pressure of the breathing gas in the respiratory line arrangement as well as
a control device for operating the breathing gas source or/and the pressure change device,
wherein the respiratory line arrangement has a respiratory line according to claim 1.

23. A breathing gas filter arrangement including a breathing gas filter accommodated in a sleeve, wherein the sleeve has a coupling formation for coupling to a coupling counter-formation of a filter carrier or of a filter heater as well as an access opening for the delivery of the breathing gas filter from the sleeve, wherein at least one wall section of the sleeve is shiftable relative to the coupling formation in such a manner that the breathing gas filter, as a result of displacement of the displaceable wall section relative to the coupling formation, is shifted through the access opening out of the sleeve.

24. The breathing gas filter arrangement according to claim 23,
wherein the access opening, in the storage state before coupling to the filter carrier or to the filter heater, is closed by means of a removable or destructible closure.
